(19) United States
(12) Patent Application Publication
(10) Pub. No.: US 2023/0200659 A1
(43) Pub. Date: Jun. 29, 2023

(54) PROXIMAL CONNECTOR FOR MEDICAL IMAGER

(71) Applicant: Sanovas Intellectual Property, LLC, Tampa, FL (US)

(72) Inventors: Alex HSIA, San Jose, CA (US); Jerry Katzman, Tampa, FL (US)

(21) Appl. No.: 18/152,729

(22) Filed: Jan. 10, 2023

Related U.S. Application Data
(63) Continuation-in-part of application No. 16/707,693, filed on Dec. 9, 2019, now Pat. No. 11,547,279.

(51) Int. Cl. A61B 5/00

(52) U.S. Cl. CPC A61B 5/0086; A61B 5/6852; A61M 25/0147

(57) ABSTRACT

An imaging catheter includes a shaft extending along a longitudinal axis between a proximal end and a distal end, a plug at the proximal end, and an imaging sensor at the distal end. The plug includes electrical terminals oriented perpendicular to the longitudinal axis. The imaging sensor includes an infrared transmitter and an infrared receiver.

### CROSS REFERENCE

The present application is a continuation-in-part of U.S. patent application Ser. No. 16/707,693, filed Dec. 9, 2019, which will issue on Jan. 10, 2023, as U.S. Pat. No. 11,547,279. The entirety of the disclosure of U.S. patent application Ser. No. 16/707,693 is hereby incorporated by reference herein.

### FIELD OF THE INVENTION

The present invention relates to medical imaging devices, such as imaging catheters and scopes. More specifically, the invention relates to such medical devices having detachable proximal ends to facilitate the removal of overlaying cannulas.

### BACKGROUND

Viewing instruments, such as a endoscopes, are generally well known in the art. Generally, an endoscope is a medical device for insertion into a body passageway or cavity that enables an operator to view and/or perform certain surgical procedures at a site inside a patient's body. The endoscope typically includes a long tubular member equipped with some type of system for transmitting images to the user, and often includes a light guide channel, channels for irrigation and suction, and a working channel for a surgical instrument. The endoscope has a proximal end that remains external to the patient, from which the operator can view the site and/or manipulate a surgical instrument, and a distal end having an endoscope tip for insertion into the body cavity of the patient.

Traditionally, these instruments have used relay optics, such as rod lenses, fiber optic bundles, or relay lenses to transmit the images from inside the body cavity of the patient to the user's eye, located at the proximal end of the endoscope, or to a camera likewise connected to the scope for subsequent display on a monitor and/storage on an image capture device.

These traditional arrangements suffer from a number of disadvantages. First, though systems for designing, constructing, and assembling relay systems have been around for some time, these systems continue to be costly, to be time-consuming, and to demand specialized expertise. Additionally, relay systems typically employ a large number of optical components, which must be precisely fabricated and positioned in order to achieve satisfactory image quality. Finally, image degradation is inevitable with such assemblies due to the fact that the light reflecting from the viewing objects must pass through a series of optical surfaces, as back-reflection, stray light, lens surface roughness, inaccuracies in lens curvatures, and slight lens misalignments all serve to reduce image quality.

Therefore, in order to attempt to circumvent these drawbacks, endoscopes employing imaging devices at their distal end were developed. For example, U.S. Pat. No. 4,074,306 to Kakinuma et al. described the use of a solid state image pick-up device at the distal end of an endoscope. Similarly, U.S. Pat. No. 4,253,447 to Moore et al. similarly disclosed the use of a solid state imaging device such as charge coupled device (CCD) at the distal end of the scope.

With the advent and refinement of solid state image sensors lie CMOS (metal-oxide semiconductor) and CCD (charge-coupled device), more expansive use of less complicated imaging catheters has also evolved. In view of the ever-increasing desire to obtain imaging devices with smaller diameters in order to view the environments within very small anatomical vessels and cavities, very thin catheters without all the channels and functionality of traditional endoscopes, but with a solid state image sensor at the distal end, have started to enjoy broader use for medical imaging.

For example, U.S. Pat. No. 8,289,381 to Bayer et al. teaches the use of an imaging catheter with a CMOS or CCD sensor at its distal tip as an auxiliary, smaller diameter imaging device within a channel of a more robust endoscope.

One shortcoming of such small diameter devices, however, is the unnecessary limitations on their use. Often, these catheters are inserted into a bodily cavity through some other, larger diameter device. This may occur, for example, when used as an auxiliary imaging device with a larger scope, as in the case of the Bayer patent discussed above, or when inserted into the body via a cannula, intubation tube, or the like. It is frequently desirable to remove the outer device, but this typically requires removing the inner imaging catheter as well. This is due to the fact that the inner imaging device is typically attached to a much wider control handle at its proximal end, which the outer shaft cannot fit over.

However, it is often desirable to leave the inner imaging device in place in the body. First, once a particular site for visualization is located, it can be difficult or time-consuming to find the site again if the imaging device is removed and then reinserted again. Additionally, it is desirable to use keep this thinner inner catheter in place while the outer cannula or other device is removed and replaced with a different cannula so that it can be used as a guidewire or “switch stick” for the new outer device.

Additionally, it also desirable to use imaging devices in which the catheter portion can be disposable without discarding the rest of the device.

For these reasons, some such devices have employed a design that renders the handle detachable. For example, U.S. Pat. No. 8,460,182 to Ouyang et al. discloses a medical instrument with a camera module at the distal tip of a cannula, which includes a CMOS, CCD, or other image sensor. The image sensor communicates with a handle connected to the proximal end of the cannula, which is detachable. In order to be detachable, the instrument has a slidable connector at its proximal end, which is plugged into a mating connector on the handle that has corresponding pin sockets for communicating with the cannula and receiving digital video signals therefrom for further transmission. As is typical, to provide this pin and socket arrangement for effectively communicating the imaging data, the slidable connector bulges radially outward, thereby resulting in a larger diameter than the rest of the cannula.

However, in order to make the imaging catheter detachable from the handle, a connector suitable for communication the imaging data from the distal image sensor must be employed. As shown in FIGS. 1A-B, this is typically in the form of a Fischer® or other pinned connecter (20), which plug into a corresponding socket (24) on the handle (26). This has several disadvantages.

First, when dealing with small diameter catheters, this connector (20) results in a bulge at the proximal end of the catheter. While smaller than the even wider handle, it still results in a larger diameter on the catheters proximal end than the rest of the catheter. Very small diameter catheters are employed for the purpose of navigating very small environments, and thus, any outer cannula used with it will likewise have a very small diameter. Yet, in order to be able to remove the outer cannula, the inner diameter of the outer cannula can only be so small, as it needs to be large enough to accommodate the connector (20) as it slides over it.

Additionally, the amount of data that can be transmitted is limited to the pins (22) that are spaced apart in the cross-sectional area of the connector (20). This, of course, is limited by the diameter of the connector (20), which cannot be too large.

What is desired, therefor, is an imaging device with a distal imager that can remain in the body when an outer cannula or other device is removed over it. What is further desired is a small diameter imaging device that can maintain the small diameter at its proximal end so that small diameter outer cannulas can be used. What is also desired is a small diameter imaging device that can communicate large amounts of data from its distal end to its handle. What is also desired is an imaging device in which the catheter portion is disposable.

### SUMMARY

One general aspect of the invention can include an imaging catheter. The imaging catheter can include a shaft extending along a longitudinal axis between a proximal end and a distal end. The catheter also includes a plug at the proximal end and the plug may include electrical terminals oriented perpendicular to the longitudinal axis. The catheter also includes an imaging sensor at the distal end. The imaging sensor may include an infrared transmitter and an infrared receiver.

Implementations may include one or more of the following features. The infrared transmitter is configured to transmit near infrared light and the infrared receiver is configured to receive the near infrared light. At least some electrical terminals of the electrical terminals are longitudinally or circumferentially offset from at least some other electrical terminals of the electrical terminals. The plug may include a curved outer wall and apertures extending through the curved outer wall, and the apertures respectively accommodate the electrical terminals. The distance sensor may include an infrared transmitter and an infrared receiver. The signal may be an amount of infrared light reflected off of the object and received at the infrared receiver of the distance sensor after emission of the infrared light from the infrared transmitter of the distance sensor. The signal may be infrared light reflected off of the object and received at the infrared receiver of the distance sensor a period of time after emission of the infrared light from the infrared transmitter of the distance sensor. The signal may be infrared light reflected off of the object at an angle and received at the infrared receiver of the distance sensor after emission of the infrared light from the infrared transmitter of the distance sensor. The signal may be infrared light reflected off of the object at a wavelength and received at the infrared receiver of the distance sensor after emission of the infrared light from the infrared transmitter of the distance sensor. The shaft further may include an inner lumen and channels and the imaging catheter may include pull wires accommodated within respective channels of the channels, and the pull wires are configured to deflect the distal end. The imaging catheter may include markers located along the longitudinal axis at the distal end.

Another general aspect of the invention can include a medical device that can include a shaft. The shaft includes extending along a longitudinal axis between a proximal end and a distal end. The medical device also includes a plug at the proximal end and the plug may include first electrical terminals oriented perpendicular to the longitudinal axis. The medical device also includes an imaging sensor at the distal end. The imaging sensor may include an infrared transmitter and an infrared receiver. The medical device also includes a handle configured to detachably connect to the proximal end of the shaft. The handle may include second electrical terminals oriented perpendicular to a longitudinal axis of the handle. When the plug is inserted in the handle, respective second electrical terminals of the second electrical terminals establish electrical connections with respective first electrical terminals of the first electrical terminal.

Implementations may include one or more of the following features. The infrared transmitter is configured to transmit near infrared light and the infrared receiver is configured to receive the near infrared light. At least some first electrical terminals of the first electrical terminals are longitudinally or circumferentially offset from at least some other first electrical terminals of the first electrical terminals, and at least some second electrical terminals of the second electrical terminals are longitudinally or circumferentially offset from at least some other second electrical terminals of the second electrical terminals. The plug may include a curved outer wall may include first apertures that respectively accommodate the first electrical terminals. The handle may include an inner wall that defines a cavity configured to receive the plug. The cavity can include second apertures that extend through the inner wall and the second apertures respectively accommodate the second electrical terminals. When the plug is inserted in the receptacle, the respective second electrical terminals are configured to be received within respective first apertures of the first apertures to establish the electrical connections. Respective electrical conductors of the electrical conductors are electrically connected to respective first electrical terminals of the first electrical terminals and to the imaging sensor.

### BRIEF DESCRIPTION OF THE DRAWINGS

FIG. 1A is a side view of prior art imaging devices.

FIGS. 1B-C are isometric views of prior art connectors used with imaging devices.

FIG. 2 is an isometric view of a system according to the invention.

FIG. 3A is a partially exposed, side view of the distal end of the imaging device of FIG. 2.

FIG. 3B is an isometric view of a camera head of the imaging device of FIG. 2.

FIG. 3C is a side view of a camera head of the imaging device of FIG. 2

FIG. 4A is an exposed, isometric view of a connector assembly used with the imaging device of FIG. 2.

FIG. 4B is an exploded, isometric view of the plug of the connector assembly of FIG. 4A.

FIG. 4C is an exploded, isometric view of the receptacle of the connector assembly of FIG. 4A.

FIG. 4D is an isometric view of the pin assembly of the receptacle of FIG. 4C.

FIG. 4E is a cross-sectional view of the connector assembly of FIG. 4A when in a connected position.

FIG. 5A is an isometric view of a connector assembly used with the imaging device of FIG. 2.

FIG. 5B is an exploded, isometric view the plug of the connector assembly of FIG. 5A.

FIG. 5C is an exploded, isometric view of part of the receptacle of the connector assembly of FIG. 5A.

FIG. 5D is an isometric view of part of the receptacle of FIG. 5C.

FIG. 5E is an exposed, side view of the connector assembly of FIG. 5A when in a connected position.

FIG. 6 is a cross-sectional view of the connector assembly of the imaging device of FIG. 2.

FIGS. 7A-E are schematic views of the operation of the imaging device of FIG. 2 in a bodily cavity.

FIG. 8 is a schematic view of the distal end of the elongated shaft of the system of FIG. 2.

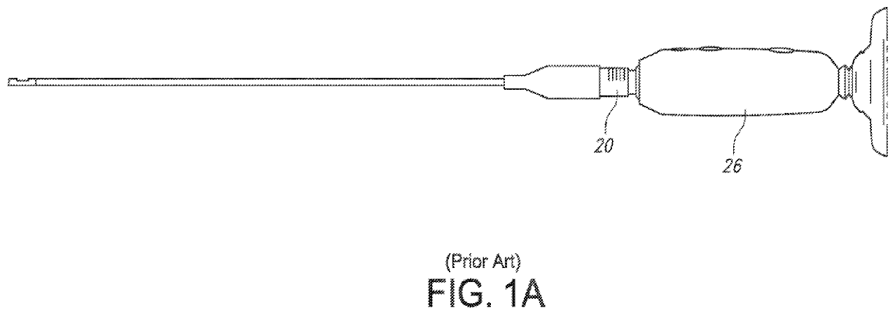

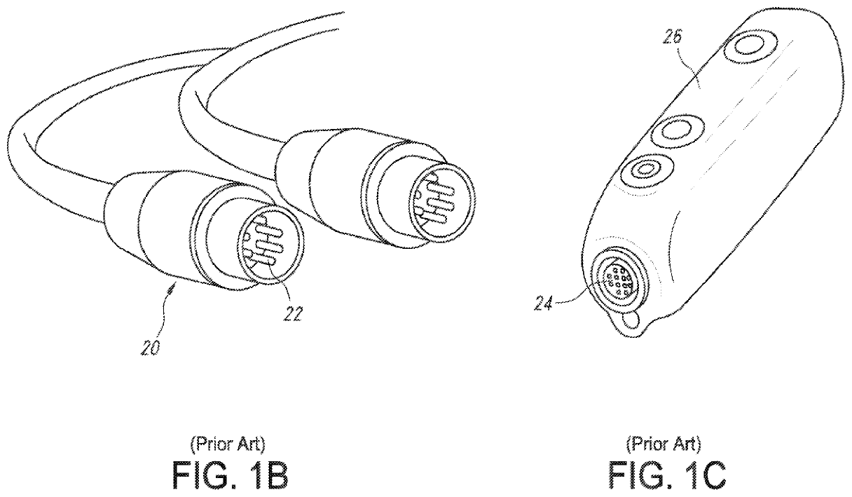

### DETAILED DESCRIPTION

The following detailed description illustrates the technology by way of example, not by way of limitation, of the principles of the invention. This description will enable one skilled in the art to make and use the technology, and describes several embodiments, adaptations, variations, alternatives and uses of the invention, including what is presently believed to be the best mode of carrying out the invention. One skilled in the art will recognize alternative variations and arrangements, and the present technology is not limited to those embodiments described hereafter.

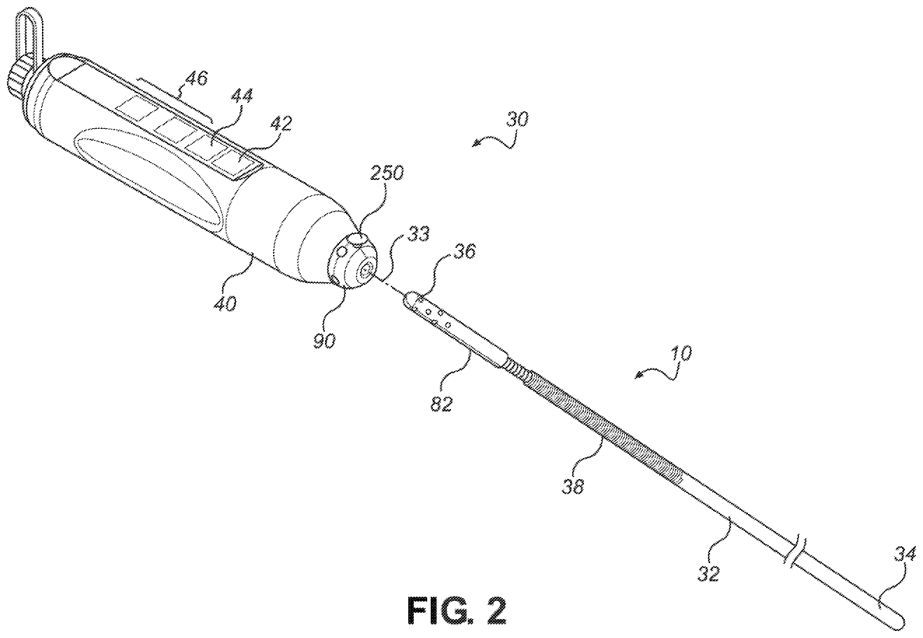

FIG. 2 illustrates one exemplary embodiment of a medical imaging device (30) in accordance with the invention. An imaging catheter includes an elongated shaft (32) having a generally cylindrical body and an inner lumen. The elongated shaft (32) has a distal end (34) for insertion into the body of a patient via an incision or natural orifice, in which the optical components of the device are housed, as further described below. The shaft (32) also has a proximal end (36) connected to a handle (40), which provides several functions. First, the handle (40) is used to hold the catheter and manipulate it in whatever bodily cavity or vessel it is inserted into. The handle (40) also typically includes basic controls associated with viewing and recording the anatomical environment, such as image capture (42), recording video (44), and adjusting the brightness (46). The handle (40) may include other controls as well, such as, for example, deflection control of the distal tip of shaft (32).

The elongated shaft (32) has a longitudinal axis (33), along which the length of the catheter is defined. The elongated shaft (32) may be constructed from any suitable rigid or semi-rigid material, such as, for example, polyether amide (PEBA), Pebax® or polyurethane. The diameter of the catheter should usually be made as small as possible.

Typically, the outer diameter of the shaft (32) is less than about 6 mm. Preferably, the outer diameter of the catheter body is less than 4 mm (e.g., 3.6 mm, 3.8 mm). In certain advantageous embodiments of the invention, the inner lumen has a diameter of at least about 1.2 mm.

An inner portion of the shaft (32) of the catheter device (10) has an inner support element (38), such as a coil, to assist the bending motion of the elongated body. The shaft (32) can be molded over during the catheter extrusion process, or the catheter body may be molded or extruded in a first step and the element (38) subsequently disposed within its inner lumen. This may comprise, for example, a coil, such as that described in U.S. Pat. No. 10,058,235 to Gunday et al. (the disclosure of which is incorporated by reference herein in its entirety), or a braided sheath, such as that disclosed in U.S. Published Application No. US 2016/0096004 by Gerrans et al. (the disclosure of which is incorporated by reference herein in its entirety). This prevents the elongated shaft (32) from kinking and provides improved torque, allowing the distal end (34) of the catheter to be stiffer while the catheter is being pushed through the bodily vessel.

In some embodiments, the shaft (32) includes at least two channels that accommodate pull wires for deflecting the distal end (34) in order to steer the catheter or adjust the angle of view at a target site. In some embodiments, the distal tip is a separate member attached to the main body of the shaft, and is fashioned from a suitable material that has a more desirable flexibility, such as, for example, a polymer plastic like polyether ether ketone (PEEK), as disclosed in U.S. Published Application No. 2016/0096004 by Gerrans et al.

In certain advantageous embodiments, the elongated shaft (32) includes imaging markers, such as radiopaque rings, located throughout the length of, or at or near, the distal end (34). Such markers can be selected and appropriately positioned in order to reflect the relevant waves of various imaging modalities (e.g., x-ray) in order to allow the use of such modalities to assist with the precise positioning of the catheter. In another advantageous embodiment, a braided sheath (discussed further below) is radiopaque.

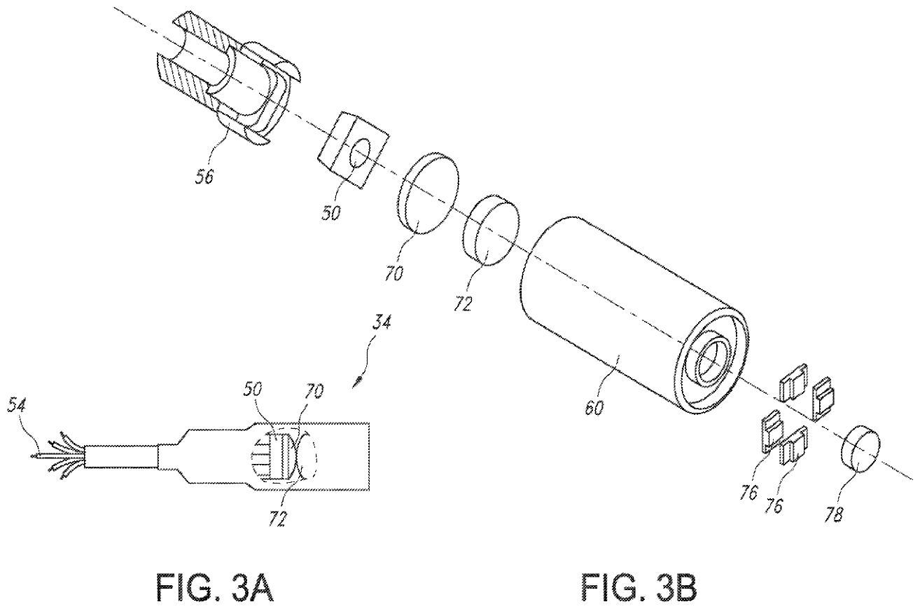

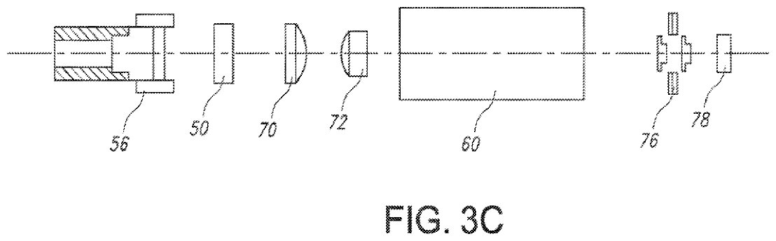

As illustrated in FIG. 3A, the distal end (34) of the elongated shaft (32) includes an image sensor (50), such as for example a solid state image sensor. In some cases, this is a CMOS (complementary metal-oxide semiconductor) sensor, and in others, this is CCD (charge-coupled device) sensor. Alternatively, a QIS (quanta image sensor) may be employed.

The image sensor (50) has a sufficiently small outer diameter, typically about 1 mm-2 mm. in some embodiments, the sensor diameter is about 0.5 mm.

A plurality of electrical conductors (54) are electrically connected to the image sensor (50), and extend down through the elongated shaft to transmit the digital signals toward the proximal end, and are electrically connected to the first plurality of electrical terminals in a plug, further described below.

Referring to FIGS. 3B-C, one advantageous embodiment of the optical components is shown. In this embodiment, a separate housing (60) connected to the distal end (34) of the shaft (32) houses various components. The housing (60) is made with any suitable material, such as plastic or metal, and has any desired shape. One or more lenses (70, 72) are positioned in the housing (60), which in the example shown, includes two positive lenses, plano-convex lenses (70) and (72), positioned opposite of each other such that the convex sides of the lenses are facing each other. It is understood that any other lens type and arrangement may be used in accordance with the present invention, as desired.

The image sensor (50) is positioned proximally of the lenses (70, 72) and coupled to a sensor mount (56) to position the image sensor (50) in the housing (60). The housing (60) also houses one or more illumination devices (76), such as LEDs, positioned distally of the lenses (70, 72). It is understood that other types of illumination devices may be used.

The distal end of the housing (60) also includes a cover glass (78), which seals the distal end of the housing (60) to protect the imaging and optical components therein.

The image sensor (50) and/or lenses (70, 72) may be oriented substantially parallel to the longitudinal axis (33) of the elongated shaft (32), or may be positioned at a certain angle relative to the longitudinal axis (33) in order to allow for better imaging of particular anatomies. In some embodiments, the image sensor (50) and/or lenses (70, 72) may be tilted to different angles by a tilting mechanism while being operated, depending on the desired angle of view, or may be advanced distally or proximally to better focus the image.

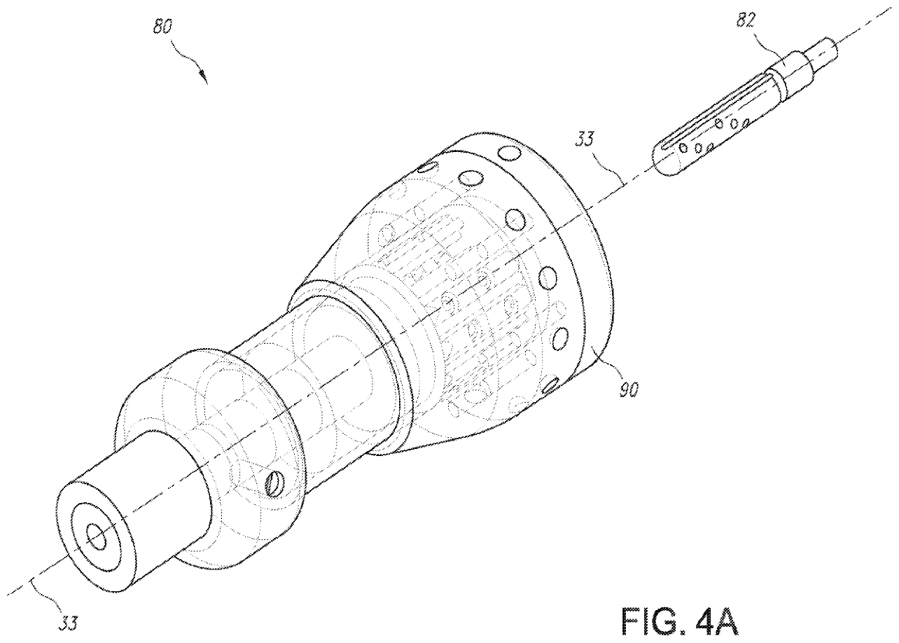

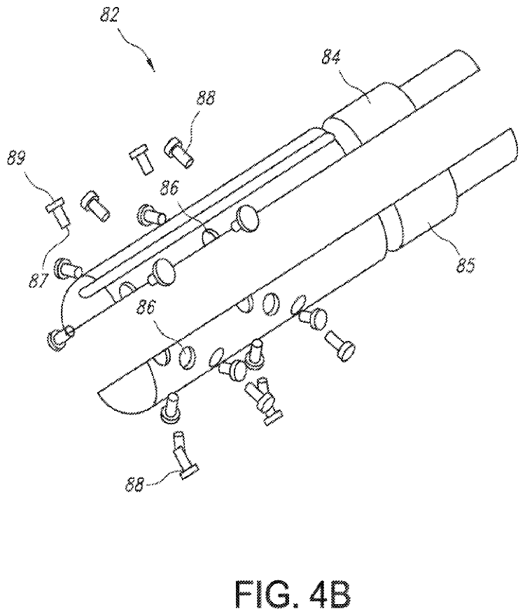

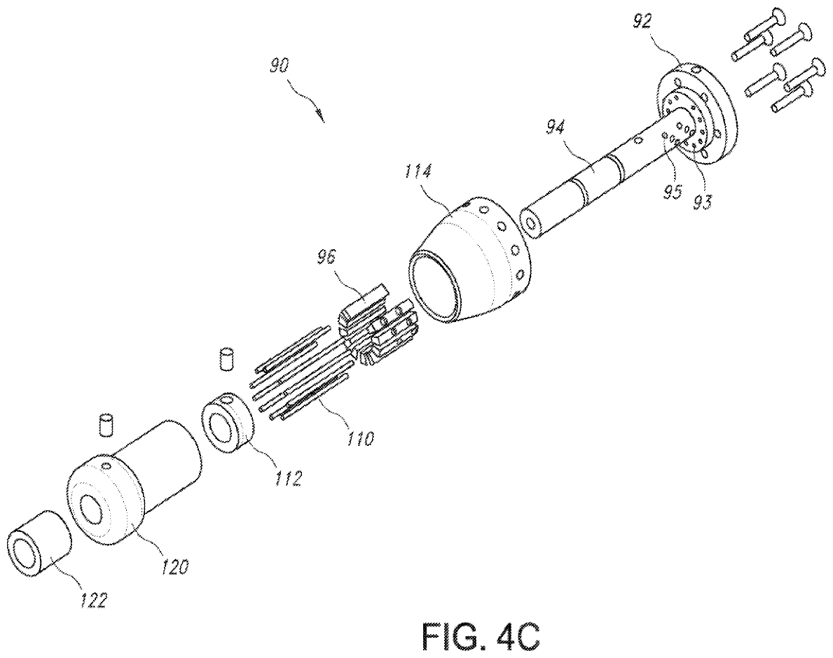

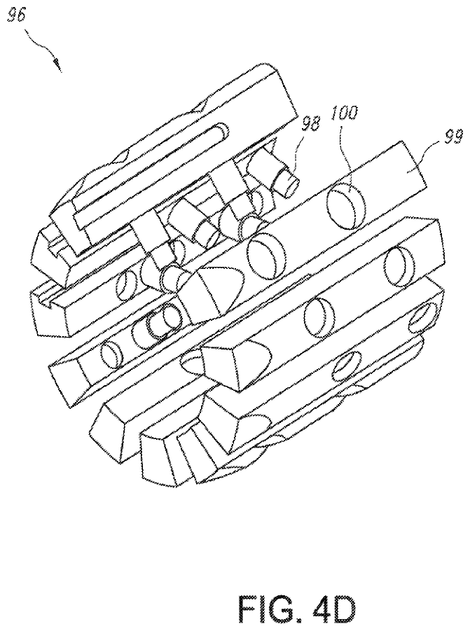

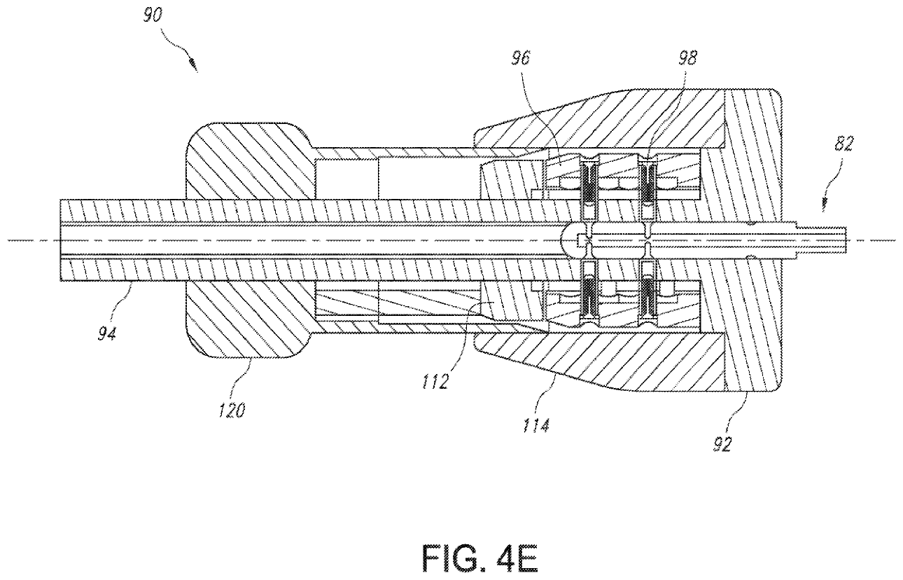

Referring to FIG. 4A, one embodiment of a connector assembly (80) by which the elongated shaft (32) is detachably connected to the handle (40) is shown. The connector assembly (80) includes a male portion in the form of a plug (82) at the proximal end (36) of the elongated shaft (32), as well as a female portion in the form of a receptacle (90).

As shown in FIG. 4B, the plug (82) is formed of first and second halves (84, 85) that are joined together and have a plurality of apertures (86) passing through the walls thereof. The apertures (86) accommodate a plurality of metallic pins (88) disposed therein. Each of the pins (88) has a tip (87) that is electrically connected (via soldering or other method) to the electrical conductors (54) extending through the elongated shaft (32), such that the electrical signals from the image sensor (50) are communicated thereto. The pins (88) extend radially from the longitudinal axis (33) of the plug (82) toward its outer circumferential wall. That is, the pins (88) can be oriented perpendicular to the longitudinal axis (33) of the plug (82). The pins (88) have an enlarged head (89) that serves as an electrical terminal by virtue of the aperture (86).

In advantageous embodiments, the pins (88) are arranged in a pattern to maximize the number of electrical terminals provided. The pins (88) are longitudinally offset from each other to take advantage of the length of the shaft (32), and the length of the plug (82) can be modified in order to increase the number of pins that can be used. Similarly, the pins (88) can be circumferentially offset from each other in order to take advantage of the circumference of the plug (82) to increase the number of pins. In the embodiment shown, the pins (88) are both longitudinally and circumferentially offset in order to maximize the number of pins that can be employed. With this arrangement, a large amount of data can be transmitted to the outer circumferential surface of the plug (82).

Referring to FIGS. 4C-D, the receptacle (90) includes a base (92) having a shaft portion (94) that extends through the length of the receptacle (90) when assembled. A pin assembly (96) containing a second plurality of pins (98) is disposed over the shaft portion (94), which has a plurality of apertures (95) through which the pins (98) will extend into the inner lumen of the shaft portion (94). The second plurality of pins (98) can be oriented perpendicular to a longitudinal axis of the handle (40). A plurality of guide pins (110) are inserted into a plurality of corresponding apertures (93) of the base (92) in order to center the pin assembly (96), and a collar (112) and outer cover (114) secure it in place. A cam lock (120) and stop cover (122) are disposed over the proximal end of the shaft portion (94).

As shown in FIG. 4D, the pin assembly (96) includes a plurality of pins (98) retained on a plurality of pin support members (99) and extending through a plurality of apertures (100) therein. Like the pins (88) of the plug (82), these pins (98) are arranged longitudinally and circumferentially offset in a pattern corresponding to that of the pins (88). The pins (98) have enlarged heads that retain them in the assembly (96), and pointed ends extending radially from the inner circumferential wall toward the center of the pin assembly (96).

As shown in FIG. 4E, the connector assembly (80) connects the proximal end of the elongated shaft (32) to the handle (40) by plugging the plug (82) into the receptacle (90). The pins (98) are spring loaded, such that, when aligned with pins (88) of the plug (82), the pins (98) descend into the apertures (86) of the plug (82) to click into place, thereby establishing an electrical connection between the pins (88) and the pins (98). As a result, a large amount of data from the image sensor (50) is communicated to the pins (98), which are themselves electrically connected to a processor in the handle (40) or some other device.

While the connecting pins (88, 98) described above have been described as all metal pins, this is not required. For example, in some embodiments, the metallic pins comprise a metal wire within an outer polymer. Other materials may be employed, as long as it is sufficient to conduct the electrical signals.

It should also be noted that, while the plug (82) is generally concentric with the elongated shaft, the outer surface of the plug (82) need not be cylindrical, and may have recesses, protuberances, undulations, and the like. Additionally, while the connector assembly (80) may be employed with an imaging catheter, it may also be employed with other narrow imaging devices, such an optical stylet, endoscope, or the like. Accordingly, the handle (40) may be a fairly sophisticated control device like that described above for use with the imaging catheter, or may simply be an outer housing of the connector assembly (80) adapted to be connected to a control box or other device.

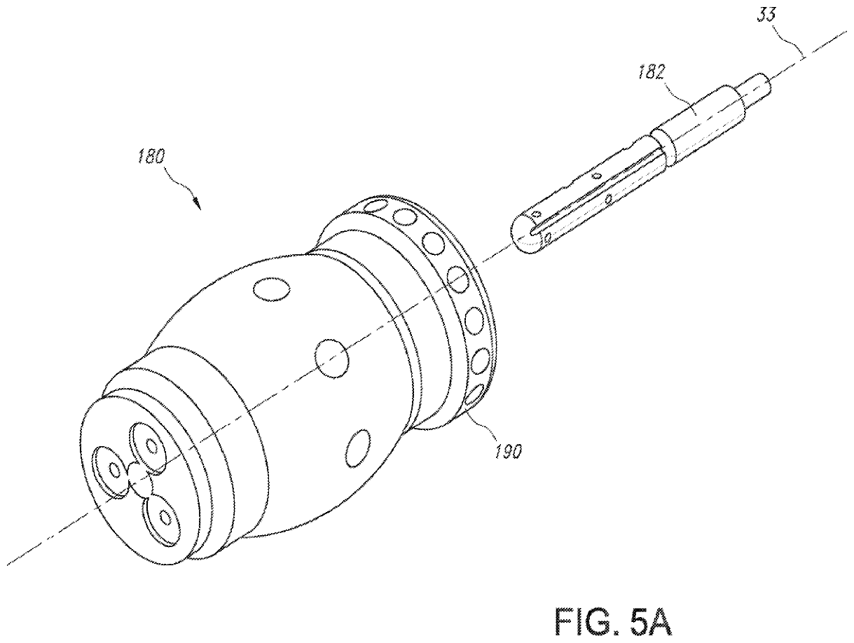

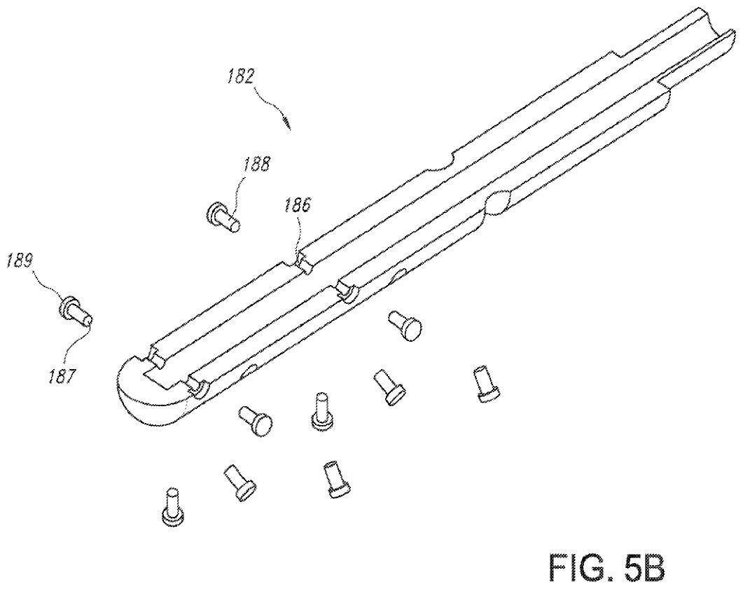

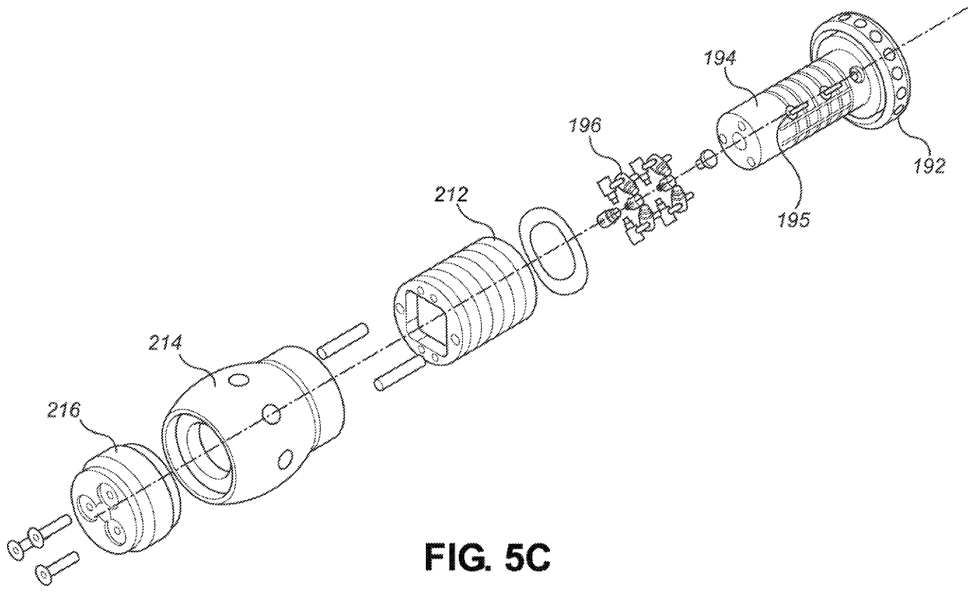

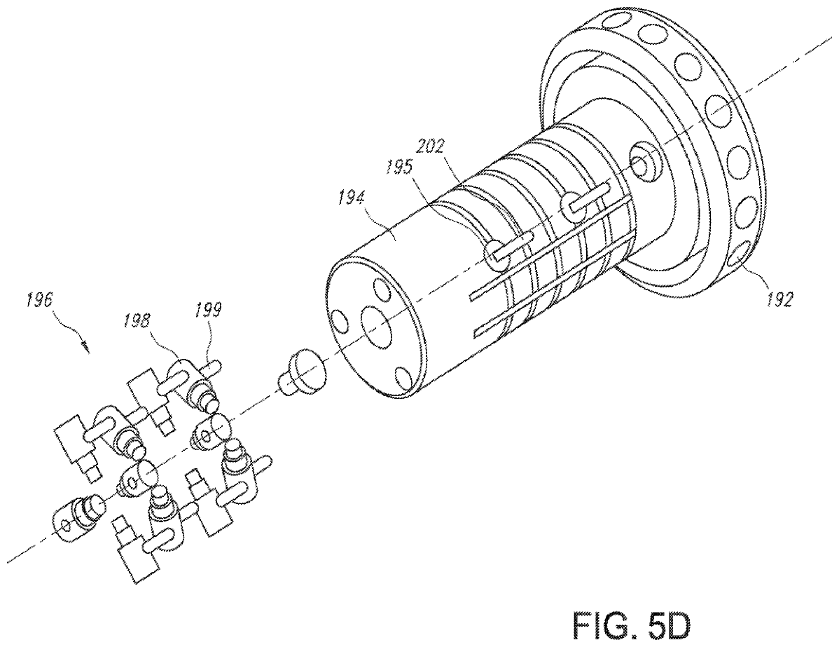

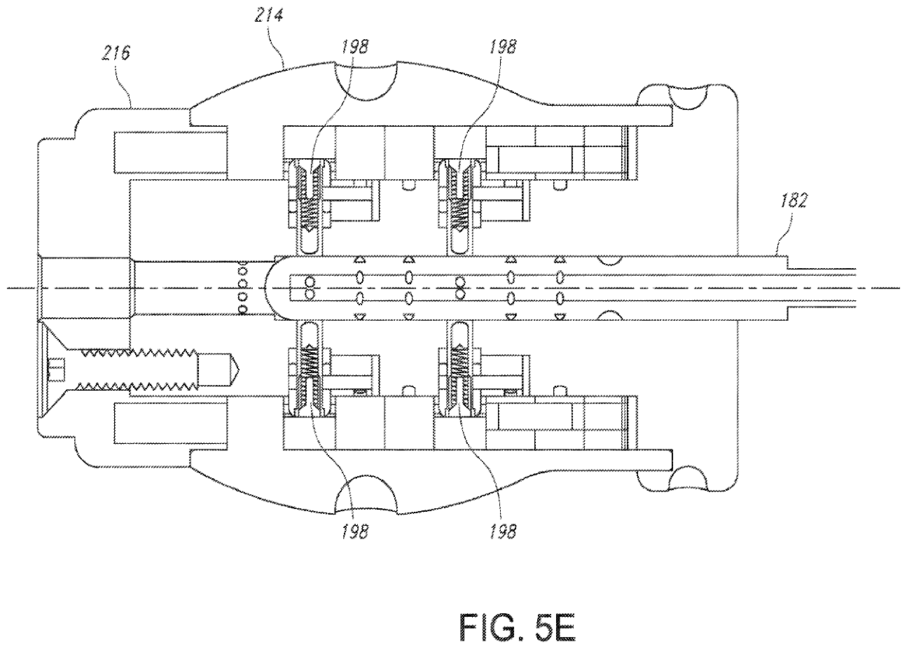

Referring to FIGS. 5A-E, another embodiment of a connector assembly (180) is shown. As shown in FIG. 5A, the connector assembly (180) includes a male plug (182) at the proximal end (36) of the elongated shaft (32), as well as a female receptacle (190).

Referring to FIG. 5B, the plug (182) similarly has a plurality of apertures (186) passing through the wall of the plug (182), which accommodate a plurality of metallic pins (188) disposed therein. Each of the pins (188) has a tip (187) that is electrically connected to the electrical conductors (54) extending through the elongated shaft (32), such that the electrical signals from the image sensor (50) are communicated thereto. The pins (188) extend radially from the longitudinal axis (33) of the plug (182) toward its outer circumferential wall, and in this case, are arranged in sequences parallel to the longitudinal axis (33), as opposed to the more curved sequences of the previous embodiment. The pins (188) have an enlarged head (189) that serves as an electrical terminal by virtue of the aperture (186). As in the previous embodiment, the pins (188) are arranged in a pattern to maximize the number of electrical terminals provided, longitudinally and circumferentially offset from each other.

Referring to FIGS. 5C-D, the receptacle (190) includes a base (192) having a shaft portion (194) that extends through the length of the receptacle (190) when assembled. A pin assembly (196) containing a second plurality of pins (198) is disposed over the shaft portion (194). The pins (198) can be oriented perpendicular to a longitudinal axis of the handle (40). The pins (198) are connected to rods (199) that sit in channels (202) of the shaft portion (194). The shaft portion (194) has a plurality of apertures (195) through which the pins (198) will extend into the inner lumen of the shaft portion (194), as previously described.

A cam assembly with locking cam (212) are connected over the pin assembly (196). A knob (214) is mounted over the cam assembly, with a collar (216) securing it in place, with which the cam assembly is operated to descend or withdraw the pins (198) in the apertures (186) to lock or release the plug (182) in the receptacle (190).

The connector assembly (180) connects the proximal end of the elongated shaft (32) to the handle (40) by plugging the plug (182) into the receptacle (190). The pins (198) are spring loaded, such that when aligned with pins (188) of the plug (182), the pins (198) contact the pins (188), establishing an electrical connection therewith.

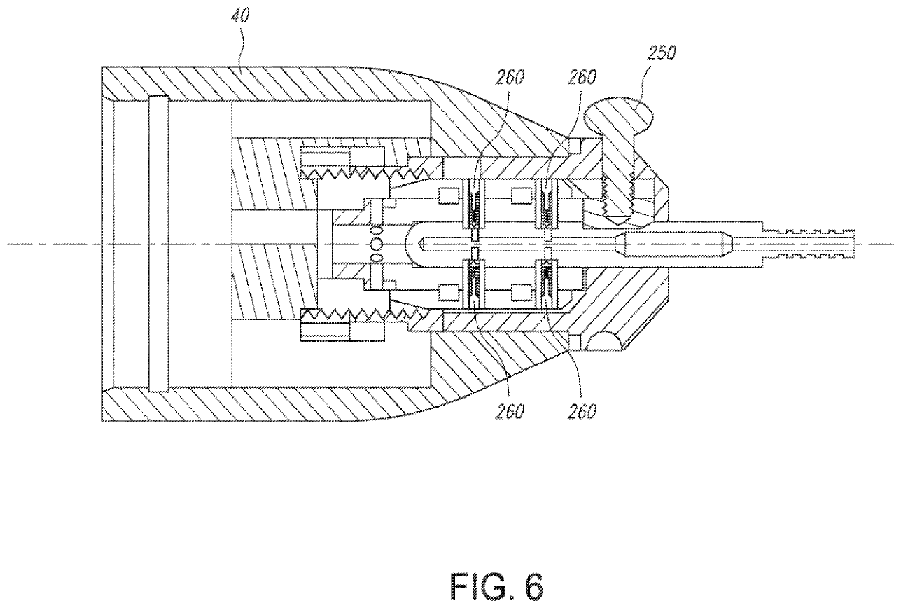

Referring to FIG. 6, in some embodiments, a button (250) is provided on the distal end of the handle (40), which can be pushed to actuate the cam mechanism that lifts the receptacle pins (260) to release the plug.

The catheter system of the present invention further includes a processor coupled to the imaging device for receiving and processing image data captured by the imaging device (30). Any suitable processor may be used in accordance with the present invention. For example, the processor may be included in the handle (40), or may be a separate device, such as a personal computer (45). In one advantageous embodiment, the processor is connected to the imaging device via a cable connection. In additional advantageous embodiments, the processor is connected to the imaging device via a wireless connection, which is desirable if a physician is located remotely from a patient being examiner or treated. Furthermore, the imaging device and/or the processor may be connected to an external storage device. The image data captured by the imaging device is stored on the storage device and may be later retrieved by a user. In other advantageous embodiments, the processor may have an internal storage device. Any suitable storage device may be used in accordance with the present invention.

The catheter system may further include a display coupled to the processor via a cable connection or via a wireless connection. The display receives imaging data processed by the processor and displays the image of the person's anatomy to a physician. Any suitable type of a display may be used in accordance with the present invention. In further advantageous embodiments, the catheter system further includes a user interface coupled to the processor. The user interface may be a graphical user interface (GUI), a keyboard, or any other suitable device that allows a user to input information and commands. The user interface is connected to the processor via a cable connection or via a wireless connection, and may be displayed on the display as on overlay image.

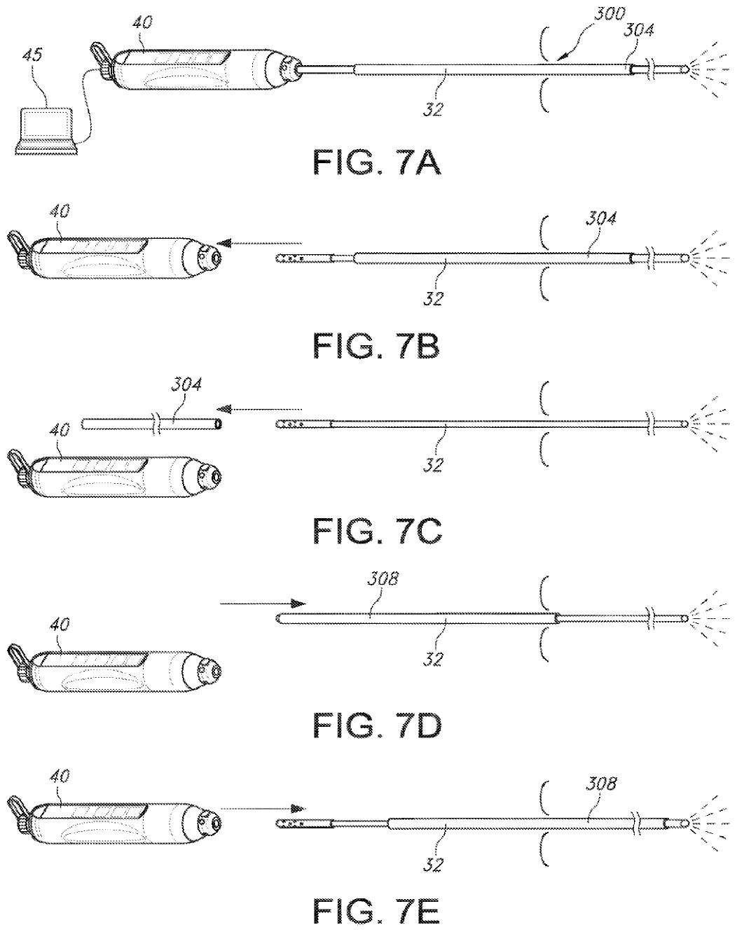

FIGS. 7A-E illustrate operation of the medical imaging device (30) of the present invention inside a bodily cavity (300). Referring first to FIG. 7A, the imaging device (30) is inserted into the bodily cavity via a surgical incision or a natural orifice through an outer cannula (304) and is then guided to the site of interest, where the image sensor obtains image data. When the user wishes to switch the outer cannula (304), the handle (40) is detached from the proximal end (36) of the shaft (32) via the connector assembly (80), as shown in FIG. 7B. The outer cannula (304) is then withdrawn from the bodily cavity over the elongated shaft (32), which remains in place, as illustrated in FIG. 7C. As shown in FIG. 7D, a new cannula (308) is advanced over the shaft (32) into the bodily cavity, using the shaft (32) as a guide. The handle (60) is then reattached to the proximal end (36) of the shaft (32) via the connector assembly (80).

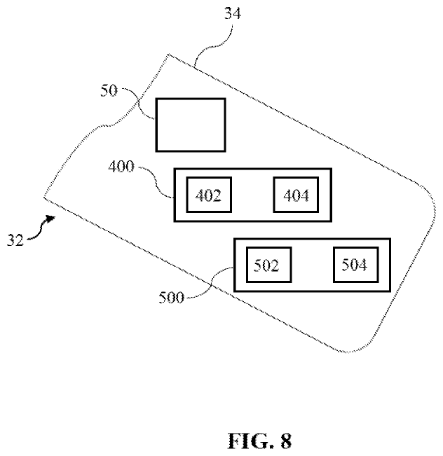

FIG. 8 illustrates a schematic view of the distal end (34) of the elongated shaft (32) of FIG. 2 according to aspects of the invention. The distal end (34) shown in FIG. 8 and described as follows can be included in any of the elongated shaft (32) embodiments previously described. As described previously, the distal end (34) can include an image sensor (50). The image sensor (50) can sense visible light (e.g., light with wavelengths from 380 to 700 nanometers). Although not shown in FIG. 8, the distal end (34) can include the housing (60), lenses (70, 72), sensor mount (56), illumination devices (76), and cover glass (78), as previously described. Additionally, the image sensor (50) can be operatively connected to and controlled by the handle (40) via the electrical conductors (54), as previously described.

The distal end (34) can include an image sensor (400). The image sensor (400) can include an infrared transmitter (402), which can illuminate a field of view of the image sensor (400) with infrared light (e.g., light with wavelengths from 0.7 to 1000 micrometers). In embodiments, the infrared transmitter (402) can illuminate the field of view with near infrared light (e.g., light with wavelengths from 0.7 to 3 micrometers).The image sensor (400) can include an infrared receiver (404), which can receive infrared light, including near infrared light. For example, the infrared receiver (404) can receive infrared light (including near infrared light) emitted from the infrared transmitter (402) and reflected back to the image sensor (400) off of objects in the field of view and the image sensor (400).

The field of view of the image sensor (400) can be directed axially out of the distal end (34) to image an environment distal to the distal end (34). The image sensor (400) can be operatively connected to the handle (40) in the same manner as described previously with respect to the image sensor (50). For example, the image sensor (400) can be operatively connected to and controlled by the handle (40) via the electrical conductors (54). The handle (40) can include a controller, as described previously, which can control the image sensor (400) to capture, record, and/or process images from the image sensor (400). Accordingly, infrared images can be captured by image sensor (400) to provide an additional or alternative view of the environment distal to the distal end (34). Since the image sensor (400) can be infrared-based, a clearer image of the environment distal to the distal end (34) can be provided and the image sensor (400) can capture images of objects even when the view of the objects are obscured, for example, by blood in the field of view of the image sensor (400). The controller of the handle (40) can be integrated directly into the handle (40) or can be an external device that is operatively connected to the handle (40).

In embodiments, the distal end (34) can include a distance sensor (500). The distance sensor (500) can detect a signal that can be used to determine a distance between the distal end (34) and an object within a field of view of the distance sensor (500). The distance sensor (500) can be an infrared sensor, an ultrasonic sensor, among other possibilities. The field of view of the distance sensor (500) can be directed axially out of the distal end (34) to detect signals that can be used to determine a distance between the distal end (34) and one or more objects distal to the distal end (34). The distance sensor (500) can be operatively connected to the handle (40) in the same manner as described previously with respect to the image sensor (50). For example, the distance sensor (500) can be operatively connected to and controlled by the handle (40) via the electrical conductors (54). The handle (40) can include a controller, as described previously, which can control the distance sensor (500) to detect signals that can be used by the controller to determine the distance between the distal end (34) and one or more objects.

In embodiments, the distance sensor (500) is an infrared image senor that can include an infrared transmitter (502) and an infrared receiver (504). The infrared transmitter (502) can emit infrared light. The infrared receiver (504) can receive infrared light. For example, the infrared receiver (504) can receive infrared light emitted from the infrared transmitter (502) and reflected back to the distance sensor (500) off of objects in the field of view of the distance sensor (500).

The handle (40) can include a controller that can determine the distance between the distal end (34) and one or more objects using the signal from the distance sensor (500). For example, in embodiments the signal from the distance sensor (500) can be an electrical signal that represents an amount of infrared light reflected off of the object and received at the infrared receiver (504) after emission of the infrared light from the infrared transmitter (502). The controller of the handle (40) can use the signal to determine the distance between the distal end (34) and the object.

In embodiments, the signal is an electrical signal that represents infrared light reflected off of the object and received at the infrared receiver (504) a period of time after emission of the infrared light from the infrared transmitter (502). Based upon the known speed of the infrared light and the period of time the controller of the handle (40) can determine the distance between the distal end (34) and the object.

In embodiments, the signal is an electrical signal that represents infrared light reflected off of the object at an angle and received at the infrared receiver (504) after emission of the infrared light from the infrared transmitter (502). The controller of the handle (40) can determine the distance between the distal end (34) and the object based on the angle of the reflected infrared light.

In embodiments, the signal is an electrical signal that represents infrared light reflected off of the object at a wavelength and received at the infrared receiver (504) after emission of the infrared light from the infrared transmitter (502). Based upon a difference between the wavelength of the emitted infrared light and the wavelength of the reflected infrared light received at the infrared receiver (504), the controller of the handle (40) can determine the distance between the distal end (34) and the object.

It should be understood that the foregoing is illustrative and not limiting, and that obvious modifications may be made by those skilled in the art without departing from the spirit of the invention. Although the invention has been described with reference to embodiments herein, those embodiments do not limit the scope of the invention. Accordingly, reference should be made primarily to the accompanying claims, rather than the foregoing specification, to determine the scope of the invention.

What is claimed is:

1. An imaging catheter comprising:
a shaft extending along a longitudinal axis between a proximal end and a distal end;
a plug at the proximal end, the plug comprising electrical terminals oriented perpendicular to the longitudinal axis; and
an imaging sensor at the distal end,
wherein the imaging sensor comprises an infrared transmitter and an infrared receiver.

2. The imaging catheter of claim 1, wherein the infrared transmitter is configured to transmit near infrared light and the infrared receiver is configured to receive the near infrared light.

3. The imaging catheter of claim 1, wherein at least some electrical terminals of the electrical terminals are longitudinally or circumferentially offset from at least some other electrical terminals of the electrical terminals.

4. The imaging catheter of claim 1, wherein the plug comprises a curved outer wall and apertures extending through the curved outer wall, and the apertures respectively accommodate the electrical terminals.

5. The imaging catheter of claim 1, further comprising a distance sensor at the distal end that is configured to detect a signal that is configured to be used to determine a distance between the distal end and an object within a field of view of the distance sensor.

6. The imaging catheter of claim 5, wherein the distance sensor comprises another infrared transmitter and another infrared receiver.

7. The imaging catheter of claim 5, wherein the signal is an amount of infrared light reflected off of the object and received at the other infrared receiver of the distance sensor after emission of the infrared light from the other infrared transmitter of the distance sensor.

8. The imaging catheter of claim 5, wherein the signal is infrared light reflected off of the object and received at the other infrared receiver of the distance sensor a period of time after emission of the infrared light from the other infrared transmitter of the distance sensor.

9. The imaging catheter of claim 5, wherein the signal is infrared light reflected off of the object at an angle and received at the other infrared receiver of the distance sensor after emission of the infrared light from the other infrared transmitter of the distance sensor.

10. The imaging catheter of claim 5, wherein the signal is infrared light reflected off of the object at a wavelength and received at the other infrared receiver of the distance sensor after emission of the infrared light from the other infrared transmitter of the distance sensor.

11. The imaging catheter of claim 1, wherein the shaft further comprises an inner lumen and channels,
the imaging catheter comprises pull wires accommodated within the channels, and
the pull wires are configured to deflect the distal end.

12. The imaging catheter of claim 1, further comprising markers located along the longitudinal axis the distal end.

13. A medical device comprising:
a shaft extending along a longitudinal axis between a proximal end and a distal end;
a plug at the proximal end, the plug comprising first electrical terminals oriented perpendicular to the longitudinal axis;
an imaging sensor at the distal end, wherein the imaging sensor comprises an infrared transmitter and an infrared receiver; and
a handle extending along a longitudinal axis and configured to detachably connect to the proximal end, the handle comprising second electrical terminals oriented perpendicular to the longitudinal axis,
wherein, when the plug is inserted in the handle, the second electrical terminals establish electrical connections with the first electrical terminals.

14. The medical device of claim 13, wherein the infrared transmitter is configured to transmit near infrared light and the infrared receiver is configured to receive the near infrared light.

15. The medical device of claim 13, wherein at least some first electrical terminals of the first electrical terminals are longitudinally or circumferentially offset from at least some other first electrical terminals of the first electrical terminals, and
wherein at least some second electrical terminals of the second electrical terminals are longitudinally or circumferentially offset from at least some other second electrical terminals of the second electrical terminals.

16. The medical device of claim 13, wherein the plug comprises a curved outer wall comprising first apertures that respectively accommodate the first electrical terminals.

17. The medical device of claim 16, wherein the handle comprises an inner wall that defines a cavity configured to receive the plug.

18. The medical device of claim 17, wherein the cavity further comprises second apertures that extend through the inner wall, wherein the second apertures respectively accommodate the second electrical terminals.

19. The medical device of claim 18, wherein, when the plug is inserted in the cavity, the second electrical terminals are received the first apertures to establish the electrical connections.

20. The medical device of claim 13, further comprising electrical conductors extending through the shaft from the proximal end to the distal end, wherein respective electrical conductors are electrically connected to respective first electrical terminals and to the imaging sensor.
